(19) United States
(12) Patent Application Publication
(10) Pub. No.: US 2023/0149218 A1
(43) Pub. Date: May 18, 2023

(54) Retractable Face Shield

(71) Applicant: Robert J. Franklin, Fort Collins, CO (US)

(72) Inventor: Robert J. Franklin, Fort Collins, CO (US)

(21) Appl. No.: 17/528,760

(22) Filed: Nov. 17, 2021

(51) Int. Cl. A61F 9/02; A41D 13/11; A42B 1/24; A62B 18/02

(52) U.S. Cl. CPC A61F 9/029; A41D 13/1184; A42B 1/24; A62B 18/02

(57) ABSTRACT

Face shields provide protection against harmful objects ranging from flying debris to bodily fluids. In the wake of the global pandemic caused by COVID-19, protective face coverings have become utilized by the general population to reduce infection. Presented herein is a novel protective face shield comprising several sections which may be articulated to operate in several positions including fully extended, fully retracted, raised, and lowered. In the fully extended position, the shield provides a level of protection for both the wearer and those around them. In the fully retracted position, the shield is raised and collapsed to conform to a typical visor.

### BACKGROUND OF THE DISCLOSURE

Protective headgear and specifically face shields have been worn by tradesmen and professionals across of various industries. Face shields provide protection against harmful objects ranging from flying debris and hot metal in fields such as construction and welding, to respiratory and bodily fluids in disciplines of medicine and dentistry. In the wake of the global pandemic caused by the spread of COVID-19, protective face coverings have gone beyond the job site or operating room and have become utilized by the general population to reduce infection.

Presented herein is a novel protective face shield which may be articulated to operate in several positions including fully extended and fully retracted. In the fully extended position, the shield provides a level of protection for both the wearer and those around them. In the fully retracted position, the shield is raised and collapsed to conform to a typical visor.

### BRIEF DESCRIPTION OF THE DRAWINGS

To facilitate a fuller understanding of the present disclosure, reference is now made to the accompanying drawings, in which like elements are referenced with like numerals. These drawings should not be construed as limiting the present disclosure but are intended to be illustrative only.

FIG. 1 shows an isometric view of a retractable face shield in the extended position.

FIG. 2 shows a front view of a retractable face shield made of several shield partitions in the extended position.

FIG. 3 shows a user wearing the face shield in the fully extend position.

FIG. 4 shows a user wearing the face shield in the fully retracted position.

FIG. 5 shows a constructed view of a retractable measuring tape assembly corresponding to an embodiment of the present disclosure.

FIG. 6 shows a side view of a sliding channel utilized for the retraction and extension of the face shield.

FIG. 7 shows a side view of the pivot channel utilized for raising and lowering the face shield.

FIG. 8 shows a side view of the face shield in the retracted position attached to the visor of a hat.

FIGS. 9A and 9B show alternative methods of attaching a face shield to a visor which may be utilized with the novel characteristics of the retractable face shield presented herein.

FIGS. 10A and 10B show alternative methods of creating a slide hinge used to bind and creating mobility between various components points of the face shield.

### DETAILED DESCRIPTION OF EMBODIMENTS

This disclosure provides detailed descriptions of inventive concepts and improvements which are applicable, but not limited to, a retractable face shield for the purpose of providing protection from debris and prevent viral transmission. As presented, the retractable face shield may be attached to an existing visor or brim of a hat or may be directly integrated into headgear such as a hard hat.

In the field, the most basic face mask is commonly attached to an uncomfortable plastic band which goes around the user's head limiting the user to either wearing the headgear or removing it. Slightly more advanced are face shields include those which flip up when the user is not in harm's way and allow eating, drinking, and unmuffled speech. A still more advanced third category exists where the face shield may be attached to a user's existing headgear thereby adding a degree of comfort and even the possibility of pivoting in order to flip up or down. While the ability to flip up and down is an improvement over the most basic face mask, a full-face mask may still awkwardly extend ten or more inches from the users face.

Presented herein is a face shield which is superior to all configurations previously identified. The face shield not only has the ability to flip up and down, but also operate between an extended position, providing full protection for the user, and retracted position, essentially minimizing the gawkiness of the face shield by hiding the shield under a visor of a hat.

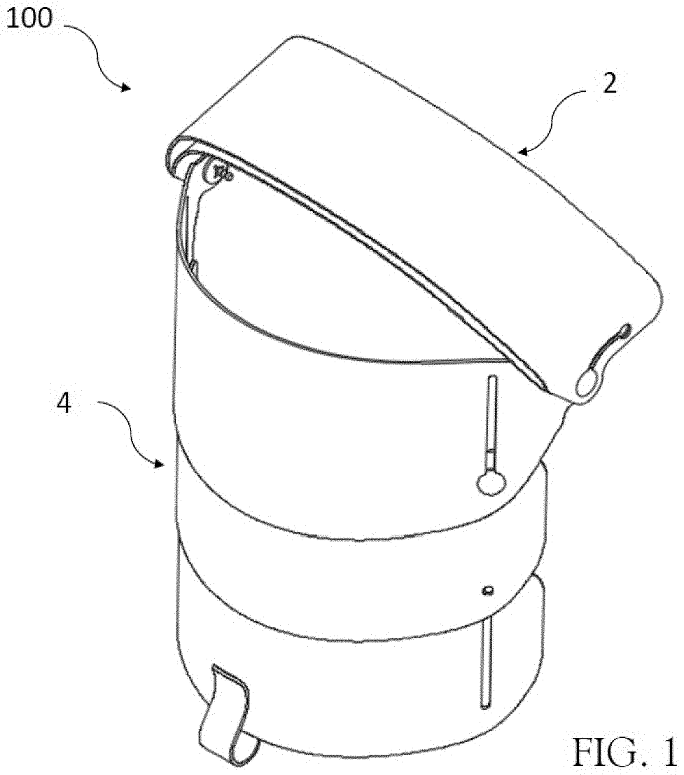

Moving to the figures, FIG. 1, the shield assembly 100 is presented as having a retractable shield 4 and a visor mount 2. As presented previously, the retractable shield may be integrated into specific headgear, however, the ability to attach to existing headgear via the visor mount 2 is presented as the preferred embodiment. Additional details on attachment to existing headgear including both a novel approach and prior art are presented within this disclosure.

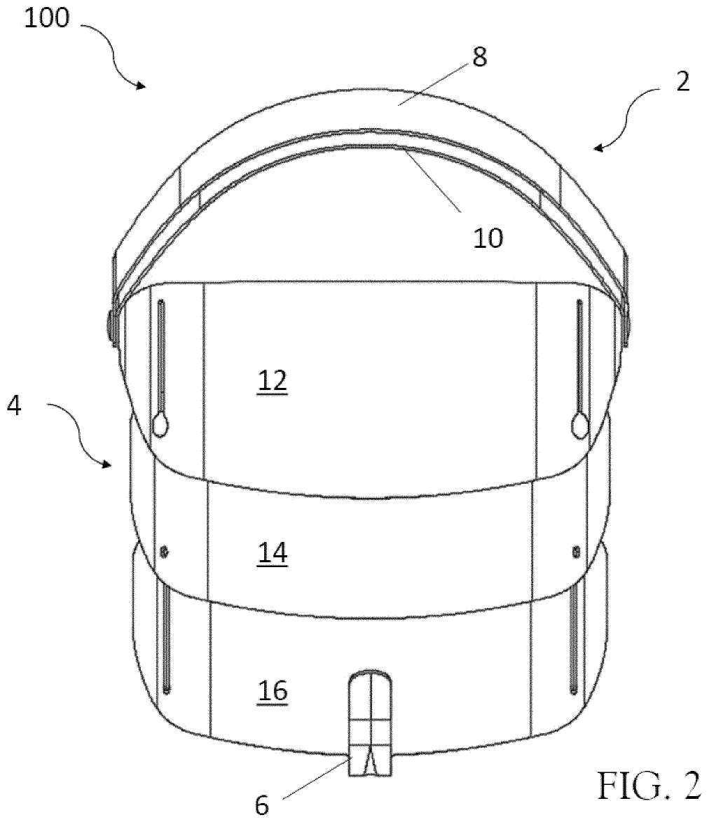

FIG. 2 shows the front view of the retractable shield 4 in the extended position as comprising three shield sections, namely the upper shield 12, the mid shield 14, and the lower shield 16. In the preferred embodiment, the lower shield includes a visor clip attached at the distal end of the lower shield. The upper shield, mid shield, and lower shield generally conform in shape to one another thereby allowing the shields to retract and extend vertically relative to the orientation of FIG. 2. To say it another way, the shields are generally constructed from a sheetlike material having a perimeter edge and a bowed parabolic surface.

The profile of each shield has a surface generally conforming to one another to allow stacking or nesting. To say it another way the surface of each shield has a shared curve profile which, when stacked, are essentially parallel. The curved profile

The visor mount 2 comprises an upper visor mount 8 and a lower visor mount 10. In the preferred embodiment, the visor or brim of the user's hat would pass and be secured between the upper and lower mount.

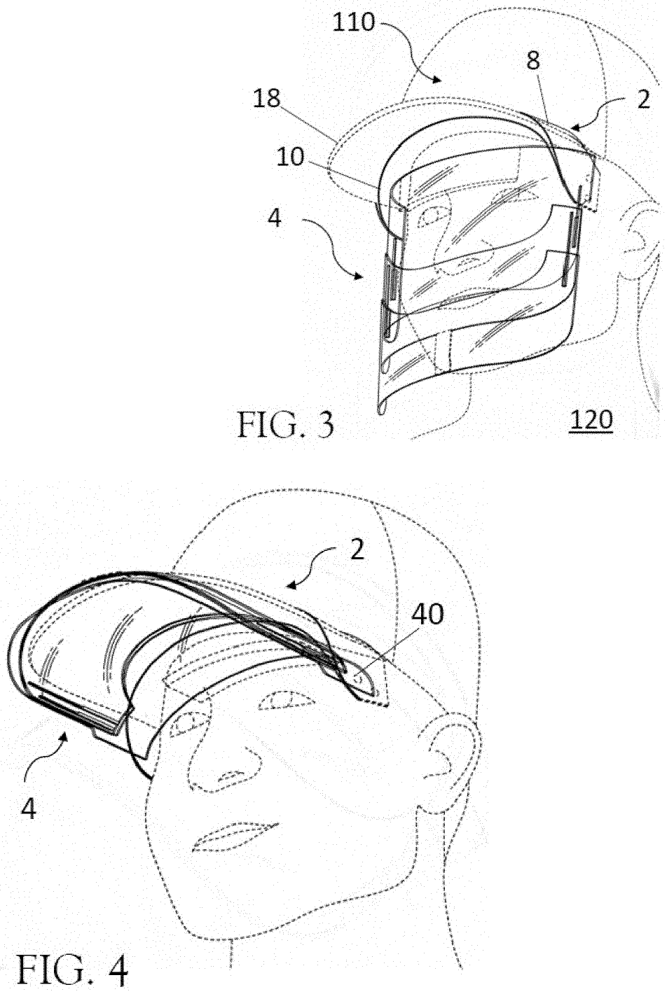

To provide a better understanding of the retractable face shield, FIG. 3 and FIG. 4 are presented to show the retractable shield 4 in the extended and retracted position.

In the fully extended position, as shown in FIG. 3, all three shield sections (i.e. upper, mid, and lower shield) are at their furthest allowable point of travel along their respective face relative to one another. Additionally, the retractable shield in FIG. 3 is shown in the flipped-down position. In the fully extended and flipped down position, the face shield provides maximum protection to the user 120. Also shown in FIG. 3 is the visor or brim 18 of the user's hat 110. As shown the visor mount 2 captures the visor between the upper visor mount 8 and lower visor mount 10.

FIG. 4 shows the retractable shield 4 in the retracted and flipped up position. In the retracted position, the three shield sections (i.e. upper, mid, and lower shield) are retracted and essentially nested within one another. To say it another way, in the retracted position, the three shields are drawn into each other. To say it yet another way, the three shields have a generally conforming profile and are coincident to each other. Additional comfort is provided to the user by being able to flip up the retractable shield 4 onto the hat brim's underside. To enable the visor to transform between a flipped-up or raised position and a down or lowered position, the retractable visor 4 is shown as being attached to the visor mount at a pivot point 40. In some embodiments, the pivot point may be on the hat or visor at a point near the edge where the visor extends from the hat.

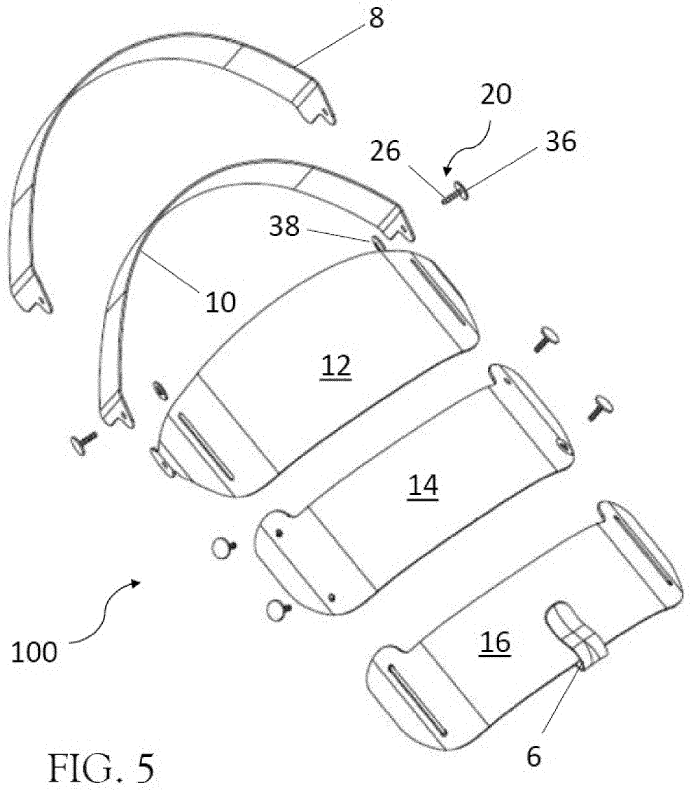

FIG. 5 shows a construction view of the complete shield assembly 100 of the preferred embodiment. Moving from top to bottom, the visor mount comprises and upper visor mount 8 and a lower visor mount 10 which are joined by a mating pair of rivets 20. The rivet 20 may consist of any of the standard fasteners having shank or body such as rivets, screws, nut & bolts, etc. In a preferred embodiment, the fastener comprises a plastic rivet 20 which includes a male rivet 36 and a mating female rivet 38. A preferred quality of the fastener is a having a fixed shank width, herein the rivet barrel 26, which secures the material between the mating parts, but does not provide total compression. That is to say, the materials secured by the rivet should have the ability to move relative to one another along a channel or groove. A plurality of rivets are shown in FIG. 5 and are provide the pivot location where the face shield may flip up or down, and along the edges of the retractable shield where the upper, mid and lower shields (12, 14, 16) may extend and retract. To say it another way, the rivet provides a protrusion that is able to travel along the channel.

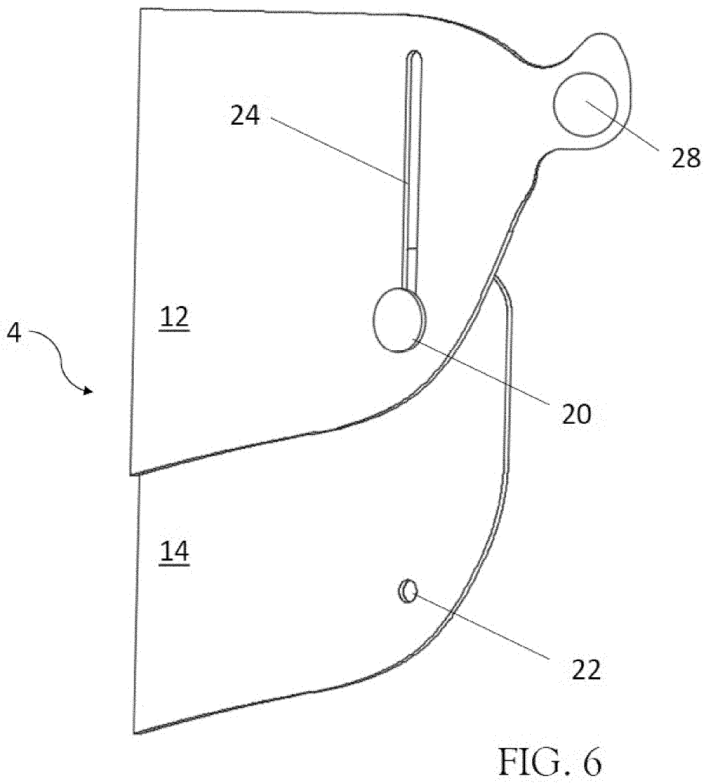

FIG. 6 is a side view of a portion of the retractable shield 4. For the purpose of explaining the vertical movement of the retractable shield (e.g., extending and retracting), only the upper shield 12 and mid shield 14 are shown. A similar configuration is used between the mid shield and lower shield. As shown, the upper shield includes a vertical slot, herein the slide channel 24, which passes through the upper shield 12. In the preferred embodiment, the position of a rivet 20 is fixed relative to the mid shield through a hole. This arrangement is also mirrored on the opposite lateral side of the retractable shield 4. The rivet 20 is able to traverse along the slide channel, thereby enabling the mid shield 14 to extend and retract relative to the upper shield 12.

While not shown in FIG. 6, the lower shield has a slide channel similar to the slide channel of the upper shield, which is also mirrored on the opposite lateral side of the retractable shield 4. This allows the lower shield to extend and retract relative to the mid shield.

Also shown in FIG. 6 is the upper pivot rivet 28. This upper pivot rivet passes through either a hole on the visor mount in the preferred embodiment, or a hole in the hat or visor, thereby enabling the flip up or flip down configuration.

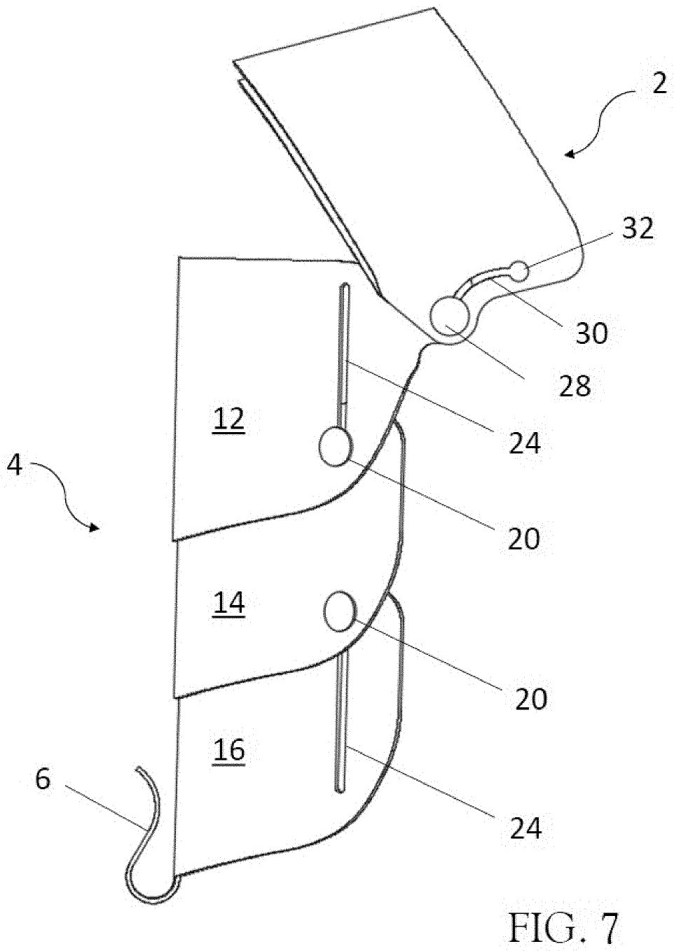

FIG. 7 presents the complete visor assembly in the fully extended and flipped down position. This figure is presented to show a preferred embodiment of the slide channel 24 interface previously described with the lower shield 16 shown. It should be understood that the slide channel and fixed rivet points may be switched or positioned interchangeable in alternative embodiments and still enable the shields traverse and nest. The preferred embodiment shown in FIG. 7 includes a configuration with two rivets 20 secured in a fixed location relative to the mid shield 14, a slide channel 24 in the upper shield 12 in which one (e.g., the upper) of the mid shield rivets passes, and a slide channel 24 in the lower shield 16 in which one (e.g., the lower) of the mid shield rivets passes. As presented earlier, the upper, mid, and lower shields are generally conforming in shape and allow the nesting, stacking, or folding into one another. Specifically, when retracted, the lower shield 16 may pass behind the mid shield 14, and the mid shield may pass behind the upper shield 12.

In alternative embodiments, the retractable face shield may comprise of two shield sections, namely an upper and lower shield. In yet another embodiment, the retractable face shield may comprise more than three shield sections.

As previously presented, the shield had the ability to pivot at a single point. FIG. 7 presents an improvement wherein the pivot rivet 28 is able to pass along a pivot channel 30 positioned on the visor mount 2. In the preferred embodiment, the pivot channel comprises an arc as shown in FIG. 7. At each end of the pivot channel is a channel lock, wherein the channel lock comprises a hole having a diameter larger than the width of the pivot channel 30. The retracted channel lock 32 resides at the proximal end of the pivot channel. The extended channel lock, not shown in FIG. 7, is positioned at the distal end of the pivot channel (under the upper pivot rivet 28).

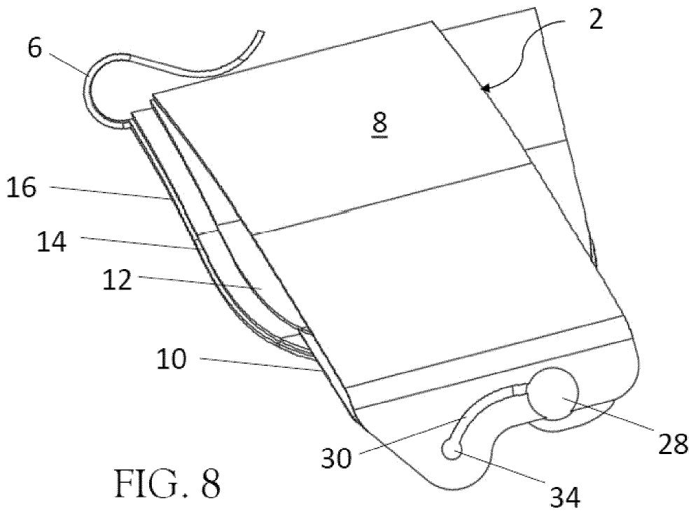

FIG. 8 presents the visor assembly in the retracted and flipped up position. In this configuration, it can be observed that the lower, mid, and upper shield (16, 14, 12) are stacked or nested on top of each other and under the visor mount 2. The upper pivot rivet 28 is secured in the retracted channel lock (not visible), and the extended channel lock 34 is shown at the distal end of the pivot channel 30.

In the retracted and flipped up position, the shield clip 6 is a retention device that provides a friction lock against the surface of the upper visor mount 8 to aid in holding this position. In some embodiments, such as those where the retractable shield is integrated directly onto a hat, the shield clip 6 may press directly against the surface of a brim or visor. In alternative embodiments, the clip may be replaced with other means to secure the retracted position such as Velcro, magnetics, or straps. The shield clip is integrated into the lower shield 16 or lowest section of the face shield.

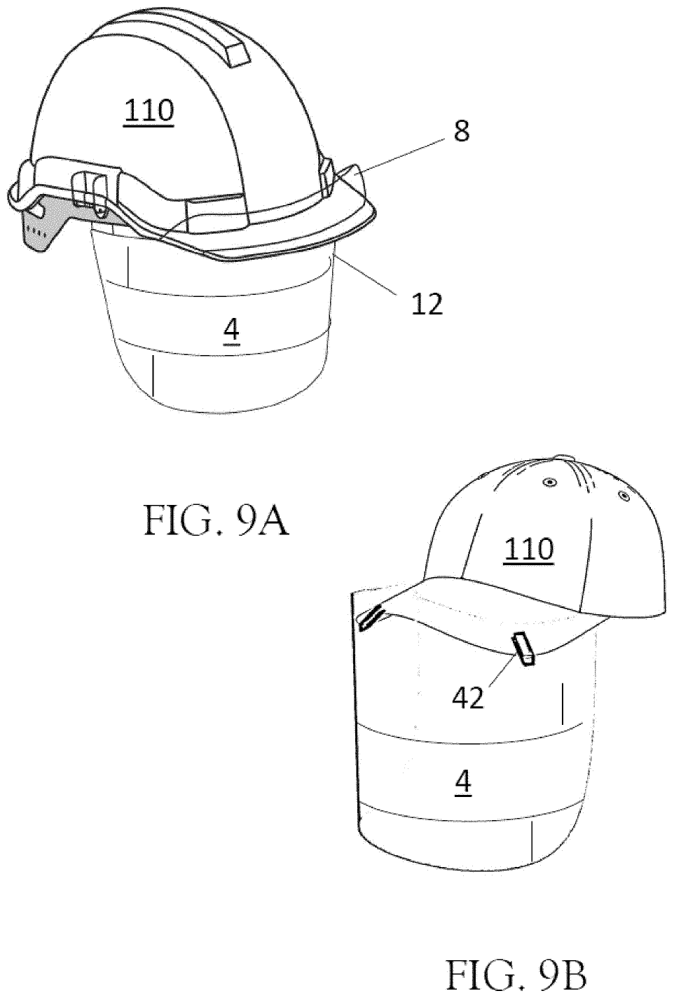

FIGS. 9A and 9B are alternative methods of mounting the retractable shield 4 to a visor or brim of existing headgear 110. In FIG. 9A, the brim of the headgear is trapped between the upper shield 12 and an upper visor mount 8 of a retractable shield 4. In FIG. 9B, the brim of the headgear is attached by clips 42 to the upper visor mount.

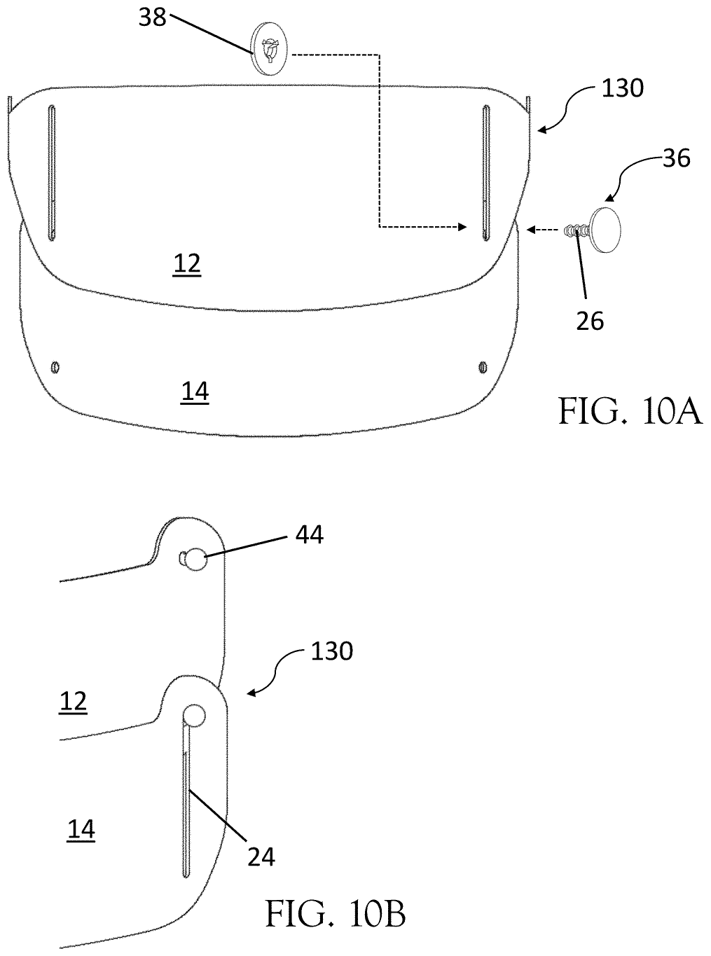

The movement points of the retractable face shield have included the barrel of a fastener (e.g. rivet barrel) passing through either a hole or channel. This arrangement is re-illustrated in FIG. 10A where two sections of the face shield are assembled using a male rivet 36 having a rivet barrel 26 and mating female rivet 38. Here the rivet barrel passes through the slide channel 24 on one section of face shield and a rivet hole 22 positioned on another section of face shield. This sliding arrangement 130 may be constructed using alternative methods including those shown in FIG. 10B. In FIG. 10B an alternative embodiment is shown where the rivet is replaced by a bulb and cylinder mechanism 44. As shown, the cylinder portion of the bulb and cylinder mechanism 44 is attached to a section of shield 12 and passes through the channel 24 on the other section of shield 14. The bulb is sized such that it may pass through the slide channel with a large amount of force (temporarily deforming either the channel or bulb). Other embodiments of the sliding arrangement may include tongue and groove sliding components such that the channel is replaced by a groove—such as a dove tail groove. In yet another embodiment of the sliding arrangement, the outside vertical edges of a shield section may traverse along a vertical slot or slide channel positioned on the face of another shield.

One unique characteristic of the slide channel positioned on a shield section is that it is linear or nearly linear, thereby allowing a secondary shield to move in a linear or vertical fashion. To say this another way, the movement is restricted in a linear orientation. This feature differentiates this design from an arced channel which may be used by alternative headgear, such as the face shield of a motorcycle helmet, where significant shield stability is required and derived from a pivot point located near the ear section of the helmet.

What is claimed is:

1. A retractable face shield which may be attached to the visor of a hat comprising
a shield and a visor mount,
wherein the shield comprises a first shield and a second shield, said first shield and second shield generally constructed of a sheetlike material having a common parabolic profile, and wherein an interface between the first and second shield comprises a sliding arrangement enabling linear movement between the first shield and second shield. shields; and
wherein the linear movement enables the shield to transform between an extended position and a retracted position.

2. The retractable face shield of claim 1, wherein the extended position is defined by the first shield positioned at a location furthest away from the second shield.

3. The retractable face shield of claim 1, wherein the retracted position is defined by the first shield positioned at a location generally coincident to the second shield.

4. The retractable face shield of claim 1, wherein a pivot point exists on the first shield which attaches to the visor mount to enable the visor to transform from a raised position to a lower position.

5. The retractable face shield of claim 4, wherein the pivot point is located near an lateral edge where the visor of the hat begins.

6. The retractable face shield of claim 4, wherein the pivot point allows the shield to be raised such that a surface of the face shield is generally coincident to the surface of the visor mount.

7. The retractable face shield of claim 4, wherein the pivot point allows the shield to flip down such that a surface of the face shield is generally perpendicular to the surface of the visor mount.

8. The retractable face shield of claim 6, wherein a retention device is positioned on either the first shield or second shield which provides a friction lock to secure the retractable face shield in the raised position.

9. A retractable face shield adapted to mount on a piece of headgear comprising:
a plurality of shields, wherein the polarity of shields have a generally conforming parabolic profile, are configured to nest within each other, and whose movement is restricted in a linear orientation along their surface.

10. A retractable face shield in a retracted position adapted to mount on a piece of headgear comprising:
a plurality of shields,
wherein each member of the plurality of shields is connected to at least one other member of the plurality of shields by a unique pair of fasteners;
wherein at least one member of the plurality of shields is nested within another member of the plurality of shields.

11. The retractable face shield of claim 9, wherein all shields within the the plurality of shields are stacked to create a top shield and a bottom shield.

12. The retractable face shield of claim 10, wherein the top shield includes a pair of upper pivot fasteners that create a hinge enabling the plurality of shields to traverse between a flipped up and flipped down orientation.

13. The retractable face shield of claim 11, wherein hinge is guided by an arced channel.

14. The retractable face shield of claim 10, wherein the bottom shield includes a retention clip which extends over the top shield.
